(19) United States
(12) Patent Application Publication
(10) Pub. No.: US 2010/0324273 A1
(43) Pub. Date: Dec. 23, 2010

(54) COMPOUNDS, COMPLEXES AND USES THEREOF

(75) Inventors: Robert D. Singer, Halifax (CA); Prashant Upendra Naik, Herouville Saint Clair (FR)

Correspondence Address: DOWELL & DOWELL P.C., 103 Oronoco St., Suite 220, Alexandria, VA, 22314, US

(73) Assignee: Saint Mary's University, Halifax (CA)

(21) Appl. No.: 12/871,049

(22) Filed: Aug. 30, 2010

Related U.S. Application Data
(63) Continuation-in-part of application No. PCT/CA2009/000229, filed on Feb. 27, 2009.
(60) Provisional application No. 61/032,103, filed on Feb. 28, 2008.

(51) Int. Cl. C07D 403/12; C07D 233/61; C07F 5/00

(52) U.S. Cl. 534/15; 548/313.7; 548/336.1; 548/106

(57) ABSTRACT

The present application relates to compounds of formulae I and II:

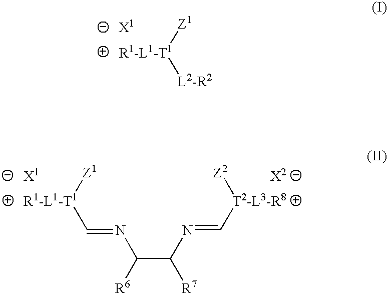

in which, X¹, X², R¹, R², R⁶, R⁷, R⁸, L¹, L², L³, T¹, T², Z¹, Z² are chosen from various possible groups or entities. For example, the compounds of formulae I and II are used as ligands for preparing complexes of various metals.

### CROSS-REFERENCE TO RELATED APPLICATIONS

This application is continuation-in-part of PCT/CA2009/000229 filed on Feb. 27, 2009, which claims priority from U.S. provisional application No. 61/032,103 filed on Feb. 28, 2008. These documents are hereby incorporated by reference in their entirety.

### TECHNICAL FIELD

There are provided improvements in the field of organic chemistry. In particular, there are provided new compounds that can be useful as ligands and ionic liquids or for forming various complexes.

### BACKGROUND OF THE APPLICATION

Ionic liquids have been defined as any ionic compound that has a melting point lower than 100° C. The use of ionic liquids as solvents has been gaining substantial interest over the last several years. These new solvents have been touted as potential “Green Solvents” for a variety of industrial applications. The usefulness of these compounds as solvents lays in a number of their physical properties that make them rather unique as compared to more traditional “molecular” or “covalent” solvents. Added functionality in ionic liquids enables them to perform specific tasks to be exploited in various applications affording what are referred to as Task Specific ionic Liquids (TSIL's).

U.S. Pat. No. 6,881,321 discloses a metal extraction process in which an ore containing a metallic element is treated with a chlorine gas so as to obtain a chloride of a metallic element. Such a chlorine metallic element is then mixed with an ionic liquid (1-butyl-methylimmidazolium chloride) so as to form an electrolyte. Finally, the metallic element is electrodeposited from the electrolyte thereby being extracted.

Several functionalities have been added to various types of ionic liquids in order to perform various specific tasks (Recent reviews include: (a) Olivier-Bourbigou, H. and Magna, L., J. Mol. Catal. A: Chem. 2002, 419, 182; (b) Wilkes, J. S., Green Chem. 2002, 4, 73; (c) Kumar, A., Chem. Rev. 2001, 101, 1; (d) Wasserschied, P. and Keim, W., Angew. Chem. Int. Ed. 2000, 39, 3772; (e) Welton, T., Chem. Rev. 1999, 99, 2071; (f) Holbrey, J. D. and Seddon, K. R., Clean Products and Processes 1999, 1, 223; (g) Olivier, H., J. Mol. Catal. A: Chem. 1999, 146, 285).

### SUMMARY OF THE APPLICATION

According to one aspect, there is included a compound of formulae (I) or (II):

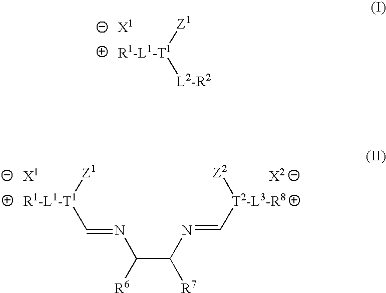

wherein
R¹ is a positively charged moiety which comprises a heteroatom and is chosen from a phosphonium derivative, a sulfonium derivative, an ammonium derivative and a positively charged heterocyclic ring, the phosphonium derivative, sulfonium derivative, ammonium derivative and the positively charged heterocyclic ring are unsubstituted or substituted with at least one substituent chosen from a halogen atom, —NO₂, —CN —OH, —CF₃ —COR^{S}, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl;
R² is ═N—OH, —C(═O)R³, —OH, —SH, —OR⁵, —SR⁵, —NH₂, NHR⁴, —N(R⁴)₂, —P(R⁵)₂, —PHR⁵₂ or —O(C═O)R³;
R³ is H, a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl;
R⁴ is a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for an amine;
R⁵ is a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for a hydroxy group, phosphine, or a thiol group;
R⁶ and R⁷ are the same or different and each represent a hydrogen atom, C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl, or R⁶ and R⁷ are joined together so as to form, for example, a C₃-C₁₂ cycloalkyl, C₁-C₁₂ heterocyclyl, C₆-C₁₂ aryl, or C₁-C₁₂ heteroaryl;

R⁸ is H or a positively charged moiety which comprises a heteroatom and is chosen from a phosphonium derivative, a sulfonium derivative, an ammonium derivative and a positively charged heterocyclic ring, the phosphonium derivative, sulfonium derivative, ammonium derivative and the positively charged heterocyclic ring are unsubstituted or substituted with at least one substituent chosen from a halogen atom, —NO₂, —CN —OH, —CF₃ —COR³, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl;
L¹ is a linker or a chemical bond;
L² is a linker or a chemical bond;
L³ is a linker or a chemical bond;
X¹ is an anion chosen from F⁻, Cl⁻, Br⁻, I⁻, ClO₄⁻, PF₆⁻, N₃⁻, BF₄⁻, SbF₆⁻, BH₄⁻, (FSO₂)₂N⁻, (CF₃SO₂)₂N⁻, (C₂F₅SO₂)₃C⁻, CF₃SO₃⁻, R³OSO₃⁻, CF₃COO⁻, AsF₆⁻, CH₃COO⁻, (CN)₂N⁻, and NO₃⁻;
X² is an anion chosen from F⁻, Cl⁻, Br⁻, I⁻, ClO₄⁻, PF₆⁻, N₃⁻, BF₄⁻, SbF₆⁻, BH₄⁻, (FSO₂)₂N⁻, (CF₃SO₂)₂N⁻, (C₂F₅SO₂)₂N⁻, (CF₃SO₂)₃C⁻, CF₃SO₃⁻, R³OSO₃⁻, CF₃COO⁻, AsF₆⁻, CH₃COO⁻, (CN)₂N⁻, and NO₃⁻;
Z¹ is —OH, —SH, —OR⁵, —SR⁵, —NH₂, —NHR⁴, —N(R⁴)₂, —PHR³, or —P(R³)₂;
Z² is —OH, —SH, —OR⁵, —SR⁵, —NH₂, —NHR⁴, —N(R⁴)₂, —PHR³, or —P(R³)₂;
T¹ is a C₆-C₁₂ aryl, partially unsaturated C₁-C₁₂ heterocyclyl or C₁-C₁₂ heteroaryl; and

T² is a C₆-C₁₂ aryl, partially unsaturated C₁-C₁₂ heterocyclyl or C₁-C₁₂ heteroaryl;
the C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl being unsubstituted or substituted with at least one substituent chosen from a halogen atom, —NO₂, —CN —OH, —CF₃ —COR³, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl.

It was found that the compounds of formulae I and II are very useful as ionic liquids and ligands. In fact, these compounds, which have ionic liquid properties, are useful for complexing or chelating metals. In an embodiment of the application, the compounds of formulae I and/or II are used in any application for which the removal of metals is required. For example, water (or other liquids and/or fluids) contaminated with metals are remediated through the use of one or more compounds of formulae I and/or II.

In a further embodiment, the compounds of formulae I and/or II form complexes with metals and these complexes serve as specialized catalysts that are soluble in other ionic liquids. In yet another embodiment, by varying the nature of the metal comprised in the complex, catalysts having different properties are obtained. In another embodiment, these catalysts are recyclable.

According to another embodiment of the present application, there is included a metal extraction process comprising contacting one or more metals with at least one compound of formula I and/or II under conditions for form a complex between the one or more metals and the at least one compounds. In an embodiment, the one or more metals are in a composition or solution comprising a solvent and the metal. In a further embodiment, the metal extraction process is used for purposes such as removing contaminating metal(s) from a composition, liquid or solution.

According to another embodiment, there is included a method for at least partially extracting one or more metals from a composition comprising the one or more metals and a liquid, the method comprising contacting the composition with at least one compound of the formula I and/or under conditions to form complexes with the one or more metals and separating the obtained complexes from the composition.

According to another embodiment, there is included a method for decontaminating a liquid that is contaminated with one or more metals, the method comprising contacting the liquid with at least one compound of the formula I and/or II under conditions to form complexes with the one or more metals separating the complexes from the liquid.

According to another embodiment, there is included a kit for extracting one or more metals comprising at least one compound of formula I and/or II, together with instructions indicating how to use the at least one compound.

According to another embodiment, there is included a kit for decontaminating a liquid contaminated with one or more metals, the kit comprising at least one compound pf the formula I and/or II, together with instructions indicating how to use the at least one compound.

It was found that such methods and kits are very useful for performing various tasks since the compounds used therewith are efficient for complexing metals.

According to another embodiment of the present application, there is included a complex comprising a metal complexed by at least one compound as those previously defined.

According to another aspect, there is included a complex comprising a metal complexed by at least two compounds of formula I and/or II In a further embodiment, the at least two compounds are the same or different.

According to another embodiment of the present application, there is included a complex of formula (III) or (IV):

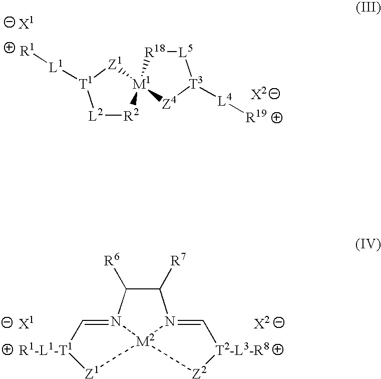

wherein
R¹ is a positively charged moiety which comprises a heteroatom and is chosen from a phosphonium derivative, a sulfonium derivative, an ammonium derivative and a positively charged heterocyclic ring, the phosphonium derivative, sulfonium derivative, ammonium derivative and the positively charged heterocyclic ring are unsubstituted or substituted with at least one substituent chosen from a halogen atom, —NO₂, —CN —OH, —CF₃ —COR³, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl;
R² is CH═N—OH, PHR⁵, —C(═O)R³, —OH, —SH, —OR⁵, —SR⁵, —NH₂, —NHR⁴, —N(R⁴)₂, P(R⁵)₂, or —O(C═O)R³;
R³ is H, a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl;
R⁴ is a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for an amine;
R⁵ is a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₃ heteroaryl, or a suitable protecting group for a hydroxy group, a phosphine, or a thiol group;
R⁶ and R⁷ are the same or different and each represent a hydrogen atom, C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl, or R⁶ and R⁷ are joined together so as to form, for example, a C₃-C₁₂ cycloalkyl, C₁-C₁₂ heterocyclyl, C₆-C₁₂ aryl, or C₁-C₁₂ heteroaryl;
R⁸ is H or a positively charged moiety which comprises a heteroatom and is chosen from a phosphonium derivative, a sulfonium derivative, an ammonium derivative, and a positively charged heterocyclic ring, the phosphonium derivative, sulfonium derivative, ammonium derivative and the positively charged heterocyclic ring are unsubstituted or substituted with at least one substituent chosen from a halogen atom, —NO₂, —CN —OH, —CF₃ —COR³, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl;
R¹⁸ is CH═N—OH, PHR⁵, —C(═O)R³, —OH, —SH, —OR⁵, —SR⁵, —NH₂, —NHR⁴, —N(R⁴)₂, P(R⁵)₂, or —O(C═O)R³;
R¹⁹ is a positively charged moiety which comprises a heteroatom and is chosen from a phosphonium derivative, a sulfonium derivative, an ammonium derivative, and a positively charged heterocyclic ring, the phosphonium derivative, sulfonium derivative, ammonium derivative and the positively charged heterocyclic ring are unsubstituted or substituted with at least one substituent chosen from a halogen atom, —NO₂, —CN —OH, —CF₃ —COR³, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl;
L¹ is a linker or a chemical bond;
L² is a linker or a chemical bond;
L³ is a linker or a chemical bond;
L⁴ is a linker or a chemical bond;
L⁵ is a linker or a chemical bond;
X¹ is an anion chosen from F⁻, Cl⁻, Br⁻, I⁻, ClO₄⁻, PF₆⁻, N₃⁻, BF₄⁻, SbF₆⁻, BH₄⁻, R³OSO₃⁻, (FSO₂)₂N⁻, (CF₃SO₂)₂N⁻, (C₂F₅SO₂)₂N⁻, (CF₃SO₂)₃C⁻, CF₃SO₃⁻, CF₃COO⁻, AsF₆⁻, CH₃COO⁻, (CN)₂N⁻, and NO₃⁻;
X² is an anion chosen from F⁻, Cl⁻, Br⁻, I⁻, ClO₄⁻, PF₆⁻, N₃⁻, BF₄⁻, SbF₆⁻, BH₄⁻, R³OSO₃⁻, (FSO₂)₂N⁻, (CF₃SO₂)₂N⁻, (C₂F₅SO₂)₂N⁻, (CF₃SO₂)₃C⁻, CF₃SO₃⁻, CF₃COO⁻, AsF₆⁻, CH₃COO⁻, (CN)₂N⁻, and NO₃⁻;
Z¹ is —OH, —SH, —OR⁵, —SR⁵, —NH₂, —NHR⁴, —N(R⁴)₂, —PHR³, or —P(R³)₂;
Z² is —OH, —SH, —OR⁵, —SR⁵, —NH₂, —NHR⁴, —N(R⁴)₂, —PHR³, or P(R³)₂;
Z⁴ is —OH, —SH, —OR⁵, —SR⁵, —NH₂, —NHR⁴, —N(R⁴)₂, —PHR³, or —P(R³)₂;
T¹ is a C₆-C₁₂ aryl, partially unsaturated C₁-C₁₂ heterocyclyl or C₁-C₁₂ heteroaryl;
T² is a C₆-C₁₂ aryl, partially unsaturated C₁-C₁₂ heterocyclyl or C₁-C₁₂ heteroaryl;
T³ is a C₆-C₁₂ aryl, partially unsaturated C₁-C₁₂ heterocyclyl or C₁-C₁₂ heteroaryl;
M¹ is a metal; and
M² is a metal;
the C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl being unsubstituted or substituted with at least one substituent chosen from a halogen atom, —NO₂, —CN —OH, —CF₃ —COR³, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl.

### BRIEF DESCRIPTION OF DRAWINGS

Further features and advantages will become more readily apparent from the following description of specific embodiments as illustrated by way of examples in the appended figures wherein:

FIG. 1 shows a X-ray crystal of a complex according to one example, wherein the complex is complex 4; and

FIG. 2 shows a X-ray crystal of a complex according to another example, wherein the complex is complex 5.

### DETAILED DESCRIPTION OF THE APPLICATION

The term “alkyl” as used herein means straight and/or branched chain, saturated alkyl groups.

The term “alkoxy” as used herein means straight and/or branched chain, saturated alkoxy groups and includes, for example, methoxy, ethoxy, propoxy, isopropoxy, n-butoxy, s-butoxy, isobutoxy, t-butoxy and the like.

The term “alkenyl” as used herein means straight and/or branched chain, unsaturated alkyl groups containing one to three double bonds, and includes, for example, vinyl, allyl, 2-methylprop-1-enyl, but-1-enyl, but-2-enyl, but-3-enyl, 2-methylbut-1-enyl, 2-methylpent-1-enyl, 4-methylpent-1-enyl, 4-methylpent-2-enyl, 2-methylpent-2-enyl, 4-methylpenta-1,3-dienyl, hexen-1-yl and the like.

The term “alkynyl” as used herein means straight and/or branched chain, unsaturated alkyl groups containing one to three triple bonds, and includes, for example, propargyl, 2-methylprop-1-ynyl, but-1-ynyl, but-2-ynyl, but-3-ynyl, 2-methylbut-1-ynyl, 2-methylpent-1-ynyl, 4-methylpent-1-ynyl, 4-methylpent-2-ynyl, 2-methylpent-2-ynyl, 4-methylpenta-1,3-diynyl, hexyn-1-yl and the like.

The term “C₃₋ₙcycloalkyl” as used herein means a monocyclic or polycyclic saturated carbocylic rings and includes, for example, cyclopropyl, cyclobutyl, cyclopentyl, cyclodecyl, bicyclo[2.2.2]octane, bicyclo[3.1.1]heptane and the like.

The term “halo” as used herein means halogen and includes chloro, fluoro, bromo and iodo.

The term “aryl” as used herein refers to a cyclic or polycyclic aromatic carbocyclic rings. In an embodiment, the aryl group is phenyl or naphthyl.

The term “heteroaryl” as used herein refers to an aromatic cyclic or polycyclic ring system having at least one heteroatom chosen from N, O, S, and P. For example, the heteroaryl groups include but are not limited to furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, among others.

The term “heterocyclyl” includes non-aromatic rings or ring systems that contain at least one ring having at least one hetero atom (such as nitrogen, oxygen, sulfur or phosphorus). For example, the heterocyclyl groups include all of the fully saturated and partially unsaturated derivatives of the above mentioned heteroaryl groups. Examples of heterocyclic groups include, without limitation, pyrrolidinyl, tetrahydrofuranyl, morpholinyl, thiomorpholinyl, piperidinyl, piperazinyl, thiazolidinyl, isothiazolidinyl, and imidazolidinyl.

The suffix “ene” added on to any of the above groups means that the group is divalent, i.e. inserted between two other groups.

The term “ring system” as used herein refers to ring structures that include monocycles, fused bicyclic and polycyclic rings, bridged rings and metalocenes.

The term “polycyclic” as used herein means groups that contain more than one ring linked together and includes, for example, groups that contain two (bicyclic), three (tricyclic) or four (quadracyclic) rings. The rings may be linked through a single bond, a single atom (spirocyclic) or through two atoms (fused and bridged).

The term “joined together” as used herein means that two substituents are linked together to form a ring system. The ring system may comprise at least 3 atoms but may also comprise several atoms, for example up to 20 atoms, which optionally includes monocyclic and polycyclic ring systems. For example, the ring system can be a C₃-C₁₂ cycloalkyl, C₁-C₁₂ heterocyclyl, C₆-C₁₂ aryl, or C₁-C₁₂ heteroaryl. According to one embodiment, the ring system can have 3 to 8, 3 to 6, 4 to 6, 5 or 6 members (or atoms).

The terms “protective group” or “protecting group” or “Pg” or the like as used herein refer to a chemical moiety which protects or masks a reactive portion of a molecule to prevent side reactions in those reactive portions of the molecule, while manipulating or reacting a different portion of the molecule. After the manipulation or reaction is complete, the protection group is typically removed under conditions that do not destroy or decompose the molecule. Many conventional protecting groups are known in the art for example as described in “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 3^{rd} Edition, 1999. These include but are not limited to t-Boc, Ts, Ms, TBDMS, TBDPS, Tf, Bn, allyl, Fmoc, C₁₋₁₆acyl, silyl, acetal and the like.

The expression “suitable protecting group for an amine” refers to a protecting group compatible with amines as defined in Greene et al. in “Protective Groups in Organic Synthesis”, 3rd Edition, 1999, 494-653, which is hereby incorporated by reference.

The expression “suitable protecting group for a hydroxy group, phosphine or a thiol group” refers to a protecting group compatible with hydroxy groups, phosphine or thiol groups as defined in Greene et al. in “Protective Groups in Organic Synthesis”, 3rd Edition, 1999, 17-292 and 454-493, which is hereby incorporated by reference.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

In an embodiment of the present application, R¹ is of the formula:

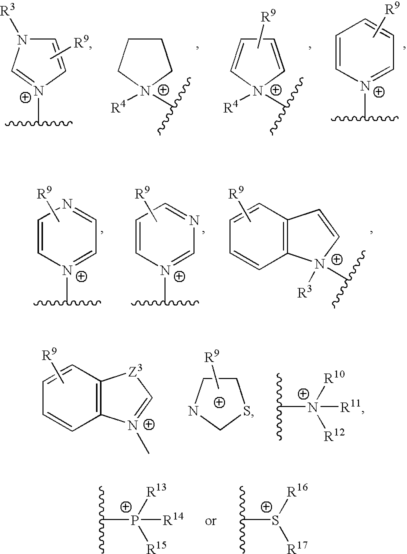

wherein
each formula is as presented above or substituted with at least one substituent as previously defined for R¹;
R⁹ represents a hydrogen atom, halogen atom, —NO₂, —CN —OH, —CF₃ —COR⁴, —SH, —OMe, —SMe, —SPh, —COOH, —COOR⁴, —NH₂, —NHR⁵, —N(R⁵)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl or C₁-C₁₂ heterocyclyl;
R¹⁰, R¹¹, and R¹² are same or different and each independently represent a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for an amine;
R¹³, R¹⁴, and R¹⁵ are same or different and each independently represent a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for a phosphorus atom;
R¹⁶ and R¹⁷ are same or different and each independently represent a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, and a suitable protecting group for a sulphur atom;
Z³ is O or S; and
R³, R⁴, and R⁵ are as previously defined.

In an embodiment of the present application, R¹ is of the formula

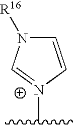

wherein R¹⁶ is a C₁-C₄ alkyl group. Methyl and butyl are non-limitative examples of such alkyl groups. In a further embodiment, R¹⁶ is alternatively a C₁-C₁₂ alkyl group. Butyl, pentyl, hexyl, heptyl, octyl, nonyl and decyl are also non-limitative examples.

In another embodiment of the present application, R¹ is a nitrogen-containing positively charged heterocyclic ring (such as imidazolium, pyridinium, pyrrolidinium, pyrimidinium, pyrazinium, or indolium).

In another embodiment of the present application, the linker groups, L¹, L², L³, L⁴ and L⁵ are a C₁-C₁₂ alkylene group.

In another embodiment, X¹ and/or X² are chosen from F⁻, Cl⁻, Br⁻, I⁻, (CN)₂N⁻, BF₄⁻, (CF₃SO₂)₂N⁻ and PF₆⁻.

In another embodiment, X¹ and/or X² are PF₆⁻.

In another embodiment, there is included a method for complexing a cation, the method comprises contacting the cation with the compounds of formula formula I and/or II.

In an embodiment, the compounds of formula I and/or II are used for complexing a cation.

In a further embodiment, the cation is a cation of a metal chosen from Li, Na, K, Rb, Cs, Fr, Be, Mg, Ca, Sr, Ba, Ra, Sc, Ti, V, Cr, Mn, Fe, Co, Ni, Cu, Zn, Y, Zr, Nb, Mo, Tc, Ru, Rh, Pd, Ag, Cd, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Ac, Th, Pa, U, Np, Pu, Am, Cm, Bk, Cf, Es, Fm, Md, No, Lr, Al, Ga, In, Ti, Sn, and Pb. In an optional embodiment, the cation is a bivalent cation. In yet another embodiment, the cation is chosen from Cu²⁺, Ni²⁺, and Co²⁺.

In yet another embodiment, the compounds of formula I and/or II are used in a metal extraction process, for purifying air, for decontaminating a liquid by extracting a metal present in the liquid with the compound, or as an ionic liquid.

In another embodiment, there is included a metal extraction process, the process comprises contacting the metal with the compounds of formula formula I and/or II so as to extract the metal.

In another embodiment, there is included a method for purifying air or decontaminating a liquid, the method comprises contacting the at least one contaminant present in the air or in the liquid with the compounds of formulae formula I and/or II. For example, the method comprises contacting the compounds of formulae I and/or II with the least one contaminant under conditions to form complexes with the one or more metals and separating the obtained complexes from the air or liquid.

In another embodiment, there is included a method of using the compounds of formula I and/or II, the method comprises carrying out a chemical reaction in which the compounds of formula I and/or II are used as an ionic liquid.

In an embodiment, the compound is a compound of formula I, and is adapted to chelate a metal so as to form a 5- or 6-membered chelate ring with said metal. In another embodiment, T¹ is phenyl and Z¹ is in the ortho position with respect to L². In yet another embodiment, -L²-R² is —CH═N—OH or -L²-R² is —COH.

In an alternate embodiment, the compound is a compound of formula II, and is adapted to chelate a metal so as to form a 5- or 6-membered chelate ring with said metal. In a further embodiment, R¹═R⁸; L¹=L³; T¹=T²; X¹═X²; and Z¹═Z². In yet another embodiment, T¹ is phenyl and Z¹ is in the ortho position with respect to the imine group. In another embodiment, the compound of formula II is a C2-symmetric chiral compound. In a further embodiment, the compound of formula II is a compound in which R⁶═R⁷═H.

It is an embodiment of the present application that, in the methods and kits previously defined, the step of extracting comprises contacting the compound formula I and/or II, in a liquid with the metals under conditions form complexes and then, separating the complexes from the liquid. In an embodiment, the cation is a cation of a metal chosen from Li, Na, K, Rb, Cs, Fr, Be, Mg, Ca, Sr, Ba, Ra, Sc, Ti, V, Cr, Mn, Fe, Co, Ni, Cu, Zn, Y, Zr, Nb, Mo, Tc, Ru, Rh, Pd, Ag, Cd, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Ac, Th, Pa, U, Np, Pu, Am, Cm, Bk, Cf, Es, Fm, Md, No, Lr, Al, Ga, In, Ti, Sn, and Pb. In another embodiment, the metal is a bivalent cation. In another embodiment, the metal is chosen from Cu²⁺, Ni²+, and Co²⁺.

In an embodiment of the application, the complexes of formulae III and IV comprise two compounds of formula I and/or II, suitably two compounds of formula I. In a further embodiment the compound of formula I and/or II are identical. In a further embodiment, the metal complexed by such compound(s) are chosen from Li, Na, K, Rb, Cs, Fr, Be, Mg, Ca, Sr, Ba, Ra, Sc, Ti, V, Cr, Mn, Fe, Co, Ni, Cu, Zn, Y, Zr, Nb, Mo, Tc, Ru, Rh, Pd, Ag, Cd, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Ac, Th, Pa, U, Np, Pu, Am, Cm, Bk, Cf, Es, Fm, Md, No, Lr, Al, Ga, In, Ti, Sn, and Pb. In yet another embodiment, the metal is a bivalent cation. In a further embodiment, the metal is chosen from Cu²⁺, Ni²+, and Co²⁺.

In an embodiment of the application, the complex is a complex of formula III, wherein Z¹-T¹-L²-R² forms a 5- or 6-membered chelate ring with M¹ and Z⁴-T³-L⁵-R¹⁸ forms a 5- or 6-membered chelate ring with M¹. In a further embodiment, T¹ is phenyl and Z¹ is in the ortho position with respect to L². In another embodiment, -L²-R² is —CH═N—OH. In another embodiment, Z¹ is —OH.

In another embodiment of the application, R¹═R¹⁹; R²═R¹⁸; L¹=L⁴; L²=L⁵; T¹=T³; X¹═X²; and Z¹═Z⁴.

In another embodiment of the application, the complex is complex of formula IV, wherein Z¹-T¹-CH═N forms a 5- or 6-membered chelate ring with M² and Z²-T²-CH═N forms a 5- or 6-membered chelate ring with M². In a further embodiment, R¹═R⁸; L¹=L³; T¹=T²; X¹═X²; and Z¹═Z². In another embodiment, T¹ is phenyl and Z¹ is in the ortho position with respect to the imine group. In a further embodiment, Z¹ is —OH.

In an embodiment of the application, the complexes of formula III and/or IV are used for purifying air or for removing metal contaminants from impure liquids such as water, factory effluent, and petroleum products.

In another embodiment, the previously defined compounds, complexes and methods also permit the quantification of the efficiency of the chelating ionic liquids for their ability. They also permit the partition of metals in to the organic phase (ICP-MS). In another embodiment, the compounds and complexes previously defined, are used to evaluate the efficiency of ionic liquids for remediation of metals ions on the environmental blacklist. In another embodiment, these compounds and complexes are reusable and are used as catalysts.

In another embodiment, the complexes of formula III and/or IV are used as catalysts. In a further embodiment, the complexes are used use as catalysts immobilized in other ionic liquids or other more conventional organic solvents.

In another embodiment, there is included a method of using the complexes of formula III and/or IV, the method comprises carrying out a chemical reaction in which the complexes of formula III and/or IV are used as a catalyst. For example, the chemical reaction is chosen from organic synthetic transformations and gas purifications.

In an embodiment, the compounds can be:

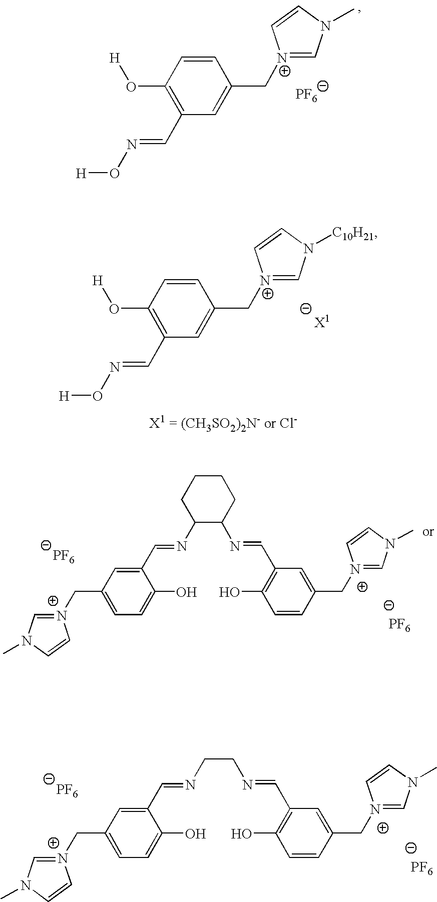

In an embodiment, the complexes can be:

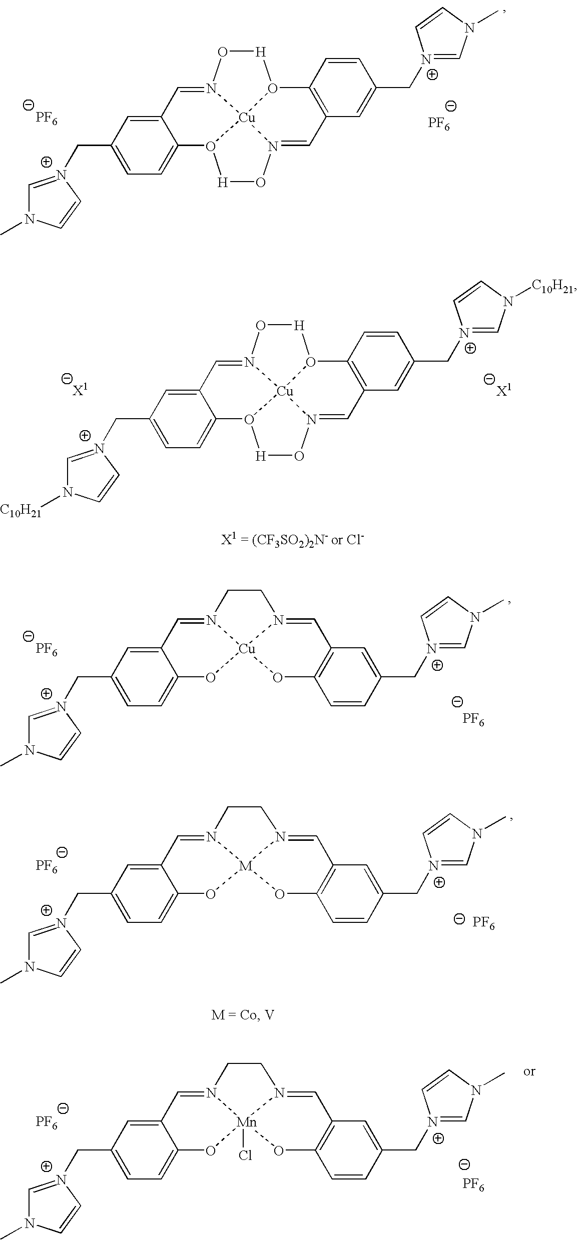

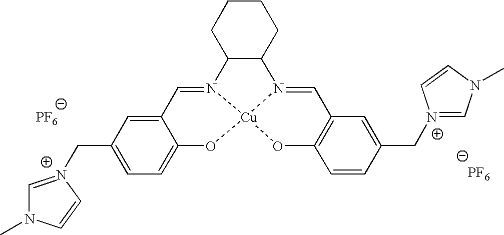

### Examples

The following non-limiting examples further illustrate various embodiments. Some examples of compounds have been prepared according to the synthetic route illustrated in Scheme 1.

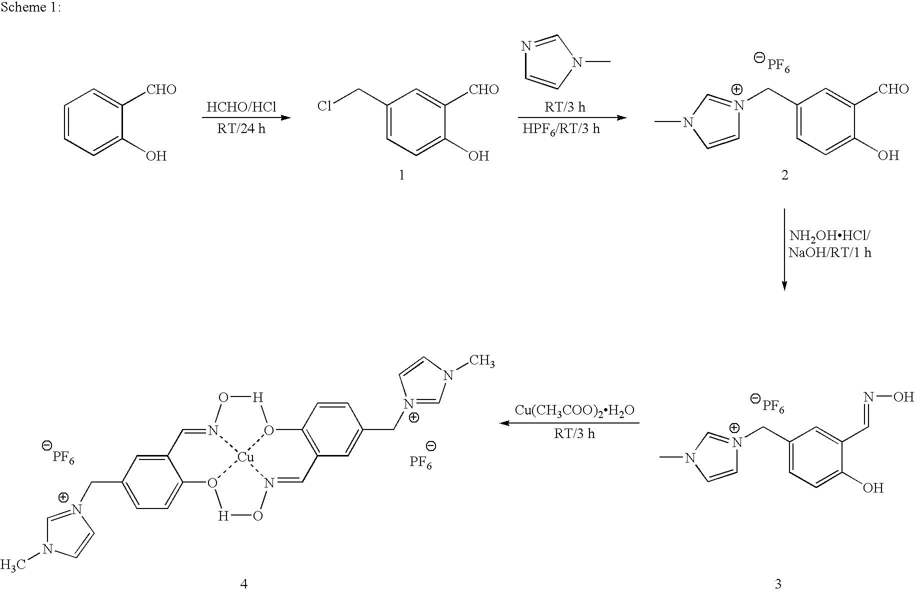

### Preparation of 5-Chloromethyl-2-hydroxybenzaldehyde (compound 1)

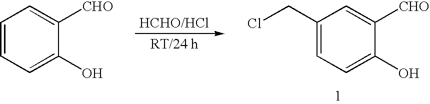

To a well stirred mixture of conc. hydrochloric acid (150 mL) and formaldehyde 37 wt % in H₂O (10.8 mL) was added salicylaldehyde (15 mL, 141.39 mmol) and the reaction was stirred at room temperature for 24 h. The solid that separated out was filtered dissolved in diethyl ether and evaporated.

Recrystallization from n-hexane afforded the product, 5-chloromethyl-2-hydroxybenzaldehyde (12.46 g) in 51.5% yield.

### Characterization Data of Compound 1:

Yield: 51.5%. IR (KBr): 3242, 3196, 2875, 2361, 1656, 1578, 1482, 1437, 1379, 1318, 1282, 1246, 1189, 1147, 1111, 948, 908, 848 cm⁻¹. ¹H NMR (250 MHz, CDCl₃): δ 4.6 (s, 2H, —CH₂—), 6.98-7.02 (d, J=8.25 Hz, 1H, 1×CH arom.), 7.54-7.60 (m, 2H, 2×CH arom.), 9.90 (s, 1H, D₂O exchangeable, Ph-OH), 11.08 (s, 1H, Ph-CHO) ppm. ¹³C NMR (63 MHz, CDCl₃): δ 45.16, 118.19, 120.22, 129.10, 133.56, 137.24, 161.48, 196.11 ppm.

### Preparation of 1-(3-formyl-4-hydroxybenzyl)-3-methylimidazolium chloride

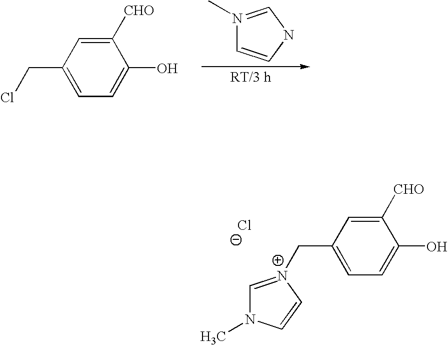

To a solution of 1-methylimidazole (7 mL, 70.17 mmol) in toluene (15 mL) at room temperature was added 5-chloromethyl-2-hydroxybenzaldehyde (10 g, 58.48 mmol) under nitrogen atmosphere. The resulting solution was stirred at room temperature for 3 h with constant stirring. The solid that separated out was washed with ether (3×10 mL) and dried to give pale yellow solid product (14.5 g) in 97% yield.

### Preparation of 1-(3-formyl-4-hydroxybenzyl)-3-methylimidazolium hexafluorophosphate (compound 2)

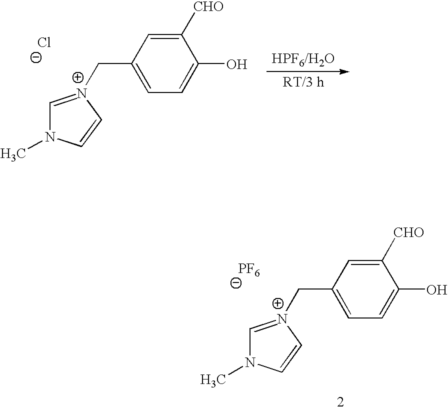

To a solution of 1-(3-formyl-4-hydroxybenzyl)-1-methylimidazolium chloride (14.8 g, 58.73 mmol) in water (100 mL) was added aqueous solution of HPF₆ (60 w % solution, 12.8 mL, 88.09 mmol) while cooling in ice bath over 1 h. After the addition was completed, the reaction was stirred at room temperature for 3 h. The solid product was filtered, washed with water and dried. The yield of the product was 22.04 g, (99%).

Characterization Data of Compound 2:

IR (KBr): 3164, 2884, 1662, 1573, 1489, 1458, 1285, 1249, 1208, 1152, 825, 759, 717, 683, 660, 623, 558 cm⁻¹. ¹H NMR (250 MHz, DMSO-d⁶): δ 3.85 (s, 3H, N—CH3—), 5.36 (s, 2H, —CH₂—), 7.04-7.08 (d, J=8.5 Hz, 1H, 1×CH arom.), 7.59-7.63 (m, 2H, 2×CH arom.), 7.69 (m, 1H, 1×CH arom.), 7.78 (m, 1H, 1×CH arom.), 9.16 (s, 1H, 1×CH arom.), 10.30 (s, 1H, D₂O exchangeable, Ph-OH), 10.98 (s, 1H, Ph-CHO) ppm. ¹³C NMR (63 MHz, DMSO-d⁶): δ 35.84, 51.09, 118.03, 122.14, 122.47, 123.98, 125.71, 128.93, 136.47, 136.60, 161.09, 190.39 ppm. ³¹P NMR (101 MHz, DMSO-d⁶): −159.43 to −117.33 ppm (septet). ¹⁹F NMR (235 MHz, DMSO-d⁶): −67.82 to −64.81 ppm (doublet). ESI MS: In positive mode peaks at m/z 217.1 (100%, [C₁₂H₁₃N₂O₂]⁺) a.m.u. and in negative mode peak at m/z 145.0 (100%, [PF₆]⁻) a.m.u.

### Preparation of 1-(3-oxime-4-hydroxybenzyl)-3-methylimidazolium hexafluorophosphate (compound 3)

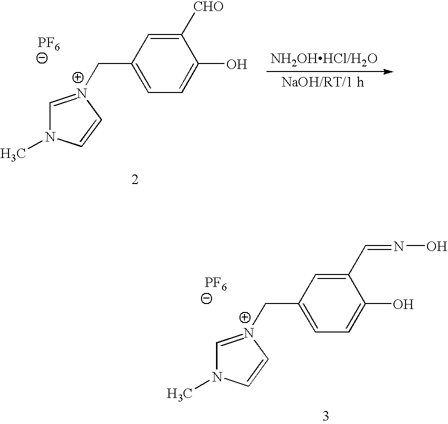

To a solution of 1-(3-formyl-4-hydroxybenzyl)-3-methylimidazolium hexafluorophosphate (10 g, 27.62 mmol) in 10 mL of ethanol was added NaOH (2.76 g, 69.05 mmol) in 25 mL of water with constant stirring at 25-30° C. To this was added drop wise hydroxylamine hydrochloride (2.28 g, 33.14 mmol) in 25 mL water. The resultant solution was stirred for 1 h. The solution was neutralized by addition of dilute HCl and the precipitate thus formed was filtered. The crude product was recrystallized by hot water and dried. The yield of the product was 5.36 g (52%).

### Characterization Data of Compound 3:

IR (KBr): 3365, 1627, 1569, 1507, 1399, 1359, 1313, 1271, 1168, 1130, 1018, 833, 674, 623, 557 cm⁻¹. ¹H NMR (250 MHz, DMSO-d⁶): δ 3.85 (s, 3H, N—CH3—), 5.32 (s, 2H, —CH₂—), 6.92-6.96 (d, J=8.25 Hz, 1H, 1×CH arom.), 7.30-7.34 (dd, J=10.5 Hz, 2H, 2×CH arom.), 7.61-7.62 (m, 1H, 1×CH arom.), 7.70 (m, 1H, 1×CH arom.), 7.76-7.77 (m, 1H, 1×CH arom.), 8.31 (s, 1H, —CH—N—OH), 9.14 (s, 1H, 1×CH arom.), 10.30 (bs, 1H, D₂O exchangeable, Ph-OH), 11.37 (bs, 1H, D₂O exchangeable, —N—OH) ppm. ¹³H NMR (63 MHz, DMSO-d⁶): δ 35.83, 51.41, 116.59, 118.88, 122.19, 123.93, 125.53, 127.61, 130.88, 136.39, 145.96, 156.15 ppm. ³¹P NMR (101 MHz, DMSO-d⁶): −159.43 to −117.35 ppm (septet). ¹⁹F NMR (235 MHz, DMSO-d⁶): −67.83 to −64.81 ppm (doublet). ESI MS: In positive mode peaks at m/z 232.1 (100%, [C₁₂H₁₃N₃O₂]⁺) a.m.u. and in negative mode peak at m/z 144.6 (100%, [PF₆]⁻) a.m.u.

### Preparation of Cu (II) Complex (Complex 4)

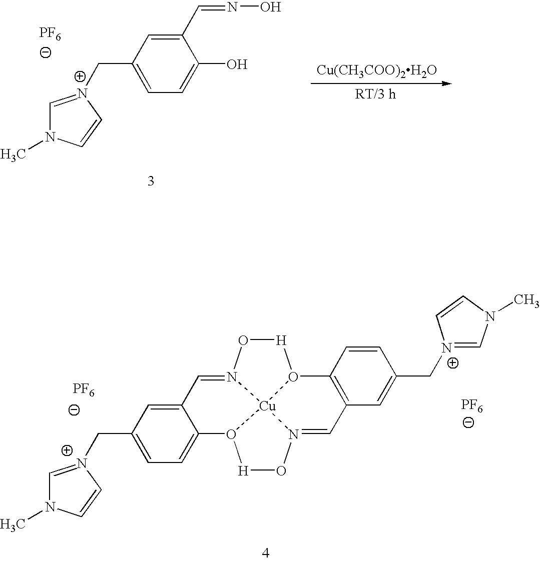

To a well stirred solution of 1-(3-oxime-4-hydroxybenzyl)-3-methylimidazolium hexafluorophosphate (0.1 g, 0.266 mmol) in 3 mL of methanol was added drop wise copper (II) acetate monohydrate (0.026 g, 0.133 mmol) dissolved in 3 mL of methanol, at room temperature. [0075] The reaction was stirred for 3 h. The precipitate formed was filtered, washed with methanol and dried. The product was recrystallized by hot methanol-water to give dark brown crystals (0.098 g) in 70% yield.

### Characterization Data of Complex 4:

IR (KBr): 3172, 3121, 1647, 1613, 1487, 1419, 1338, 1304, 1166, 1027, 825, 750, 670, 615, 553 cm⁻¹. HR-ESI-MS: In positive mode peaks at 670.0961 a.m.u. [⁶³Cu(C₂₄H₂₆N₆O₄PF₆)] and 672.0961 a.m.u. [⁶⁵Cu(C₂₄H₂₆N₆O₄PF₆)], and in negative mode peak at 145 a.m.u.([PF₆]⁻).

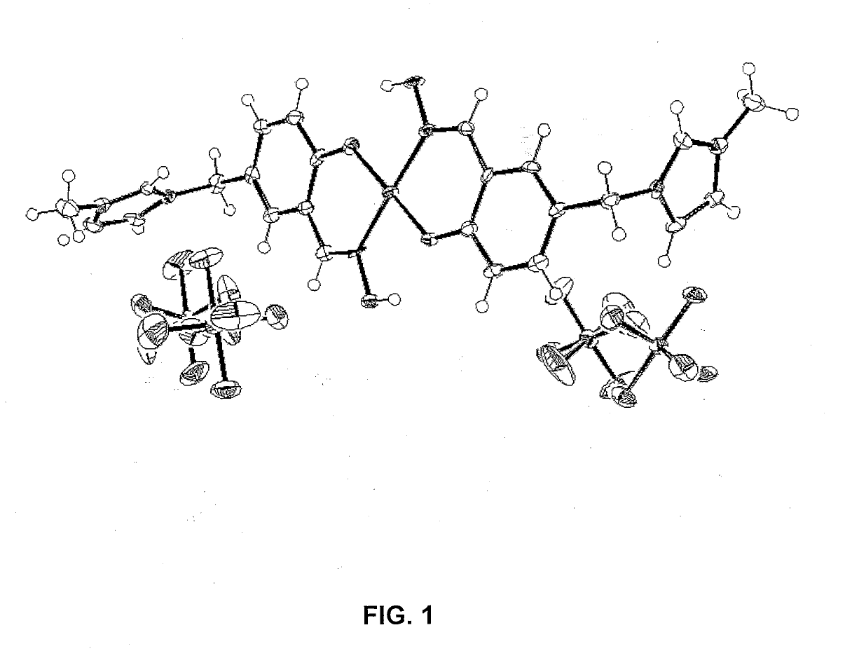

### The X-Ray Crystal Structure is Shown in FIG. 1.

Further examples of compounds have been prepared according to the synthetic route illustrated in Scheme 2.

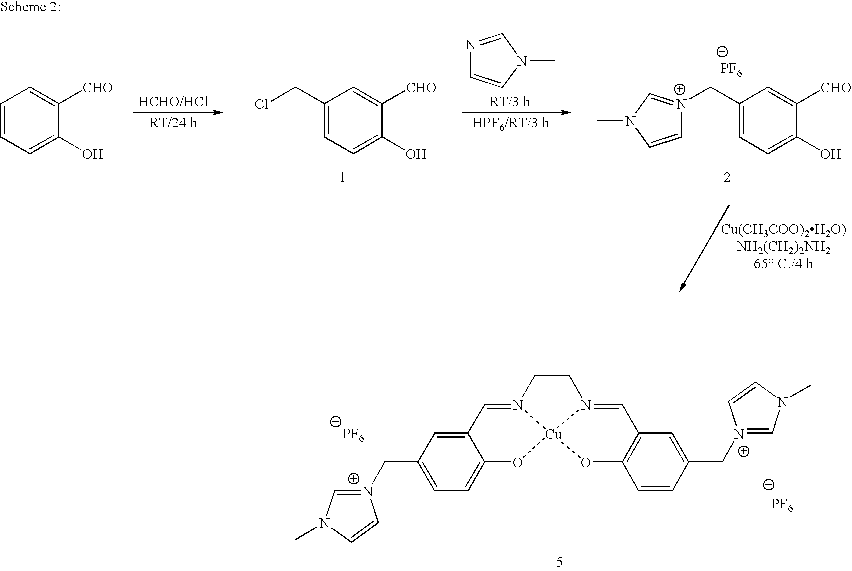

### Preparation of Salen-Copper Complex (Complex 5):

To a well stirred solution of Cu(CH₃COO)₂. H₂O (0.054 g, 0.276 mmol) in 100 mL of dry methanol, under reflux, were added separately and simultaneously the solution of 1-(3-formyl-4-hydroxybenzyl)-3-methylimidazolium hexafluorophosphate 2 (0.100 g, 0.276 mmol) in 5 mL of methanol and ethylenediamine (0.016 g, 0.276 mmol) in 5 mL of methanol by a syringe pump over 30 minutes. The mixture was refluxed for 4 h. After cooling the reaction mixture was concentrated under reduced pressure to a volume about 10 mL and kept overnight at 0° C. Under these conditions, the product precipitated was filtered from the mother liquor and dried. The yield of the product was 0.175 g, 78.5%.

IR (KBr): 3168, 1635, 1571, 1536, 1477, 1392, 1310, 1220, 1163, 1087, 835, 751, 620, and 555 cm⁻¹. HR-ESI-MS: In positive mode peaks at 664.1185 a.m.u. [⁶³Cu(C₂₆H₂₈N₆O₂PF₆)] and in negative mode peak at 145 a.m.u.([PF₆]⁻)

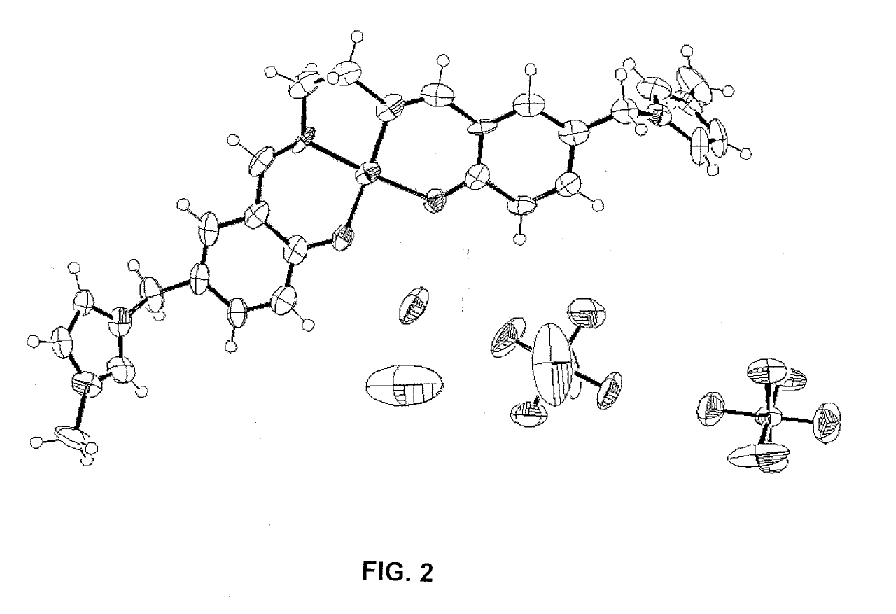

The X-ray crystal structure is shown in FIG. 2. The structure of FIG. 2 comprises two water molecules.

The compounds defined in the present document can be used for similar purposes as the compounds described in WO 2007/071028.

While specific embodiments have been described, it will be understood that the technology hereby presented can be further modified and this application is intended to cover any variations, uses, or adaptations thereof.

What is claimed is:

1. A compound of formula (I) or (II): 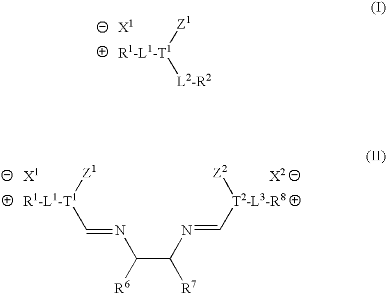
wherein
R¹ is a positively charged moiety which comprises a heteroatom and is chosen from a phosphonium derivative, a sulfonium derivative, an ammonium derivative and a positively charged heterocyclic ring, the phosphonium derivative, sulfonium derivative, ammonium derivative and the positively charged heterocyclic ring are unsubstituted or substituted with at least one substituent chosen from a halogen atom, —NO₂, —CN —OH, —CF₃ —COR^{S}, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl;
R² is ═N—OH, —C(═O)R³, —OH, —SH, —OR⁵, —SR⁵, —NH₂, —NHR⁴, —N(R⁴)₂, —P(R⁵)₂, —PHR⁵₂ or —O(C═O)R³;
R³ is H, a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ alkylaryl, or C₁-C₁₂ heteroaryl;
R⁴ is a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for an amine;
R⁵ is a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for a hydroxy group, phospine, or a thiol group;
R⁶ and R⁷ are the same or different and each represent a hydrogen atom, C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl;
R⁸ is a positively charged moiety which comprises a heteroatom and is chosen from a phosphonium derivative, a sulfonium derivative, an ammonium derivative and a positively charged heterocyclic ring, the phosphonium derivative, sulfonium derivative, ammonium derivative and the positively charged heterocyclic ring are unsubstituted or substituted with at least one substituent chosen from a halogen atom, —NO₂, —CN —OH, -CF₃ —COR³, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl;
L¹ is a linker or a chemical bond;
L² is a linker or a chemical bond;
L³ is a linker or a chemical bond;
X¹ is an anion chosen from F⁻, Cl⁻, Br⁻, I⁻, ClO₄⁻, PF₆⁻, N₃⁻, BF₄⁻, SbF₆⁻, BH₄⁻, (FSO₂)₂N⁻, (CF₃SO₂)₂N⁻, (C₂F₅SO₂)₂N⁻, (CF₃SO₂)₃C⁻, CF₃SO₃⁻, R³OSO₃⁻, CF₃COO⁻, AsF₆⁻, CH₃COO⁻, (CN)₂N⁻, and NO₃⁻;
X² is an anion chosen from F⁻, Cl⁻, Br⁻, I⁻, ClO₄⁻, PF₆⁻, N₃⁻, BF₄⁻, SbF₆⁻, BH₄⁻, (FSO₂)₂N⁻, (CF₃SO₂)₂N⁻, (C₂F₅SO₂)₂N⁻, (CF₃SO₂)₃C⁻, CF₃SO₃⁻, R³OSO₃⁻, OF₃COO⁻, AsF₆⁻, CH₃COO⁻, (CN)₂N⁻, and NO₃⁻;
Z¹ is —OH, —SH, —OR⁵, —SR⁵, —NH₂, —NHR⁴, —N(R⁴)₂, —PHR³, or —P(R³)₂;
Z² is —OH, —SH, —OR⁵, —SR⁵, —NH₂, —NHR⁴, —N(R⁴)₂, —PHR³, or —P(R³)₂;
T¹ is a C₆-C₁₂ aryl, partially unsaturated C₁-C₁₂ heterocyclyl or C₁-C₁₂ heteroaryl; and
T² is a C₆-C₁₂ aryl, partially unsaturated C₁-C₁₂ heterocyclyl or C₁-C₁₂ heteroaryl;
said C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl being unsubstituted or substituted with at least one substituent chosen from a halogen atom, —NO₂, —CN —OH, —CF₃ —COR³, —SH, —OMe, —SMe, —SPh, —COOH, —COOR⁴, —NH₂, —NHR⁴, —N(R⁴)₂, C₂^{-C}₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl.

2. The compound of claim 1, wherein R¹ is a nitrogen-containing positively charged heterocyclic ring.

3. The compound of claim 1, wherein R¹ is of formula: 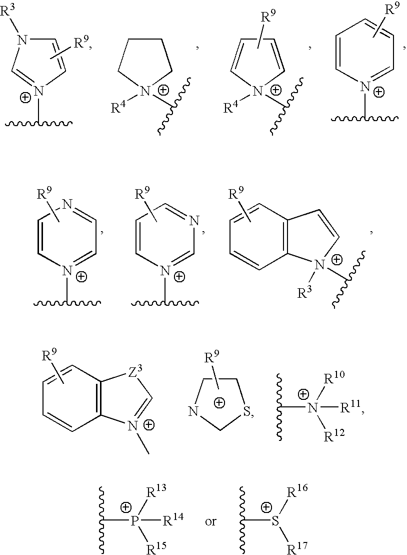
wherein
each formula is as presented above or substituted with at least one substituent as defined for R¹ in claim 1;
R⁹ represents a hydrogen atom, halogen atom, —NO₂, —CN —OH, —CF₃ —COR⁴, —SH, —OMe, —SMe, —SPh, —COOH, —COOR⁴, —NH₂, —NHR⁵, —N(R⁵)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl or C₁-C₁₂ heterocyclyl;
R¹⁰, R¹¹, and R¹² are same or different and each independently represent a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for an amine;
R¹³, R¹⁴, and R¹⁵ are same or different and each independently represent a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for a phosphorus atom;
R¹⁶ and R¹⁷ are same or different and each independently represent a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, and a suitable protecting group for a sulphur atom;
Z³ is O or S; and
R³, R⁴, and R⁵ are as previously defined in claim 1.

4. The compound of claim 1, wherein R¹ is of formula: 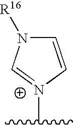
wherein R¹⁶ is a C₁-C₁₂ alkyl group.

5. The compound of claim 4, wherein R¹⁶ is methyl, butyl, hexyl, octyl, or decyl.

6. The compound of claim 4, wherein L¹ is a C₁-C₁₂ alkyl group.

7. The compound of claim 4, wherein said compound is a compound of formula (I), said compound being adapted to chelate a metal so as to form a 5- or 6-membered chelate ring with said metal, and T¹ is a phenyl and wherein Z¹ is in ortho position with respect to L², -L²-R² being —CH═N—OH.

8. The compound of claim 1, wherein said compound is a compound of formula (II), said compound being adapted to chelate a metal so as to form a 5- or 6-membered chelate ring with said metal, R¹═R⁸; L¹=L³; T¹=T²; X¹═X²; and Z¹═Z², and wherein T¹ is a phenyl and wherein Z¹ is in ortho position with respect to the imine group.

9. The compound of claim 8, wherein Z¹ is —OH.

10. The compound of claim 6, wherein X¹ is chosen from F⁻, Cl⁻, Br⁻, I⁻, (CN)₂N⁻, BF₄⁻, (CF₃SO₂)₂N⁻ and PF₆⁻.

11. The compound of claim 4, wherein X¹ is chosen from Cl⁻, (CF₃SO₂)₂N⁻ and PF₆⁻.

12. A method for at least partially extracting a metal from a composition comprising said metal and a liquid, said method comprising reacting said composition with at least one compound as defined in claim 1 so as to form a complex and separating the obtained complex from the rest of said composition.

13. A compound of formula (I) or (II): 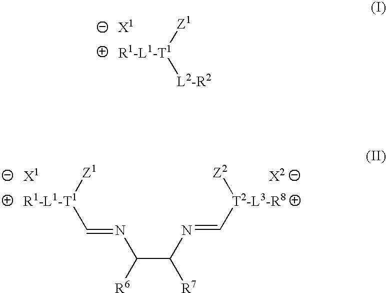
wherein
R¹ is a positively charged moiety which comprises a heteroatom and is chosen from a phosphonium derivative, a sulfonium derivative, an ammonium derivative and a positively charged heterocyclic ring, the phosphonium derivative, sulfonium derivative, ammonium derivative and the positively charged heterocyclic ring are unsubstituted or substituted with at least one substituent chosen from a halogen atom, —NO₂, —CN —OH, —CF₃ —COR³, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl;
R² is ═N—OH, —C(═O)R³, —OH, —SH, —OR⁵, —SR⁵, —NH₂, —NHR⁴, —N(R⁴)₂, —P(R⁵)₂, —PHR⁵₂ or —O(C═O)R³;
R³ is H, a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl;
R⁴ is a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for an amine;
R⁵ is a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for a hydroxy group, phospine, or a thiol group;
R⁶ and R⁷ are the same or different and each represent a hydrogen atom, C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl, or R⁶ and R⁷ are joined together so as to form a C₃-C₁₂ cycloalkyl, C₁-C₁₂ heterocyclyl, C₆-C₁₂ aryl, or C₁-C₁₂ heteroaryl;
R⁸ is a positively charged moiety which comprises a heteroatom and is chosen from a phosphonium derivative, a sulfonium derivative, an ammonium derivative and a positively charged heterocyclic ring, the phosphonium derivative, sulfonium derivative, ammonium derivative and the positively charged heterocyclic ring are unsubstituted or substituted with at least one substituent chosen from a halogen atom, —NO₂, —CN —OH, —CF₃ —COR³, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl;
L¹ is a linker or a chemical bond;
L² is a linker or a chemical bond;
L³ is a linker or a chemical bond;
X¹ is an anion chosen from F⁻, Cl⁻, Br⁻, I⁻, ClO₄⁻, PF₆⁻, N₃⁻, BF₄⁻, SbF₆⁻, BH₄⁻, (FSO₂)₂N⁻, (CF₃SO₂)₂N⁻, (C₂F₅SO₂)₂N⁻, (CF₃SO₂)₃C⁻, CF₃SO₃⁻, R³OSO₃⁻, OF₃COO⁻, AsF₆⁻, CH₃COO⁻, (CN)₂N⁻, and NO₃⁻;
X² is an anion chosen from F⁻, Cl⁻, Br⁻, I⁻, ClO₄⁻, PF₆⁻, N₃⁻, BF₄⁻, SbF₆⁻, BH₄⁻, (FSO₂)₂N⁻, (CF₃SO₂)₂N⁻, (C₂F₅SO₂)₂N⁻, (CF₃SO₂)₃C⁻, CF₃SO₃⁻, R³OSO₃⁻, OF₃COO⁻, AsF₆⁻, CH₃COO⁻, (CN)₂N⁻, and NO₃⁻;
Z¹ is —OH, —SH, —OR⁵, —SR⁵, —NH₂, —NHR⁴, —N(R⁴)₂, —PHR³, or —P(R³)₂;
Z² is —OH, —SH, —OR⁵, —SR⁵, —NH₂, —NHR⁴, —N(R⁴)₂, —PHR³, or —P(R³)₂;
T¹ is a C₆-C₁₂ aryl, partially unsaturated C₁-C₁₂ heterocyclyl or C₁-C₁₂ heteroaryl; and
T² is a C₆-C₁₂ aryl, partially unsaturated C₁-C₁₂ heterocyclyl or C₁-C₁₂ heteroaryl;
said C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl being unsubstituted or substituted with at least one substituent chosen from a halogen atom, —NO₂, —CN —OH, —CF₃ —COR³, —SH, —OMe, —SMe, SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl.

14. A complex comprising a metal complexed by two compounds of formula (I) or by a compound of formula (II) as defined in claim 13.

15. The complex of claim 14, wherein said metal is chosen from Li, Na, K, Rb, Cs, Fr, Be, Mg, Ca, Sr, Ba, Ra, Sc, Ti, V, Cr, Mn, Fe, Co, Ni, Cu, Zn, Y, Zr, Nb, Mo, Tc, Ru, Rh, Pd, Ag, Cd, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Ac, Th, Pa, U, Np, Pu, Am, Cm, Bk, Cf, Es, Fm, Md, No, Lr, Al, Ga, In, Ti, Sn, and Pb.

16. The complex of claim 14, wherein said metal is chosen from V, Mn, Co, Ni, and Cu.

17. The complex of claim 14, wherein said metal is chosen from Cu²⁺, Ni²⁺, and Co²⁺.

18. A complex of formula (III) or (IV): 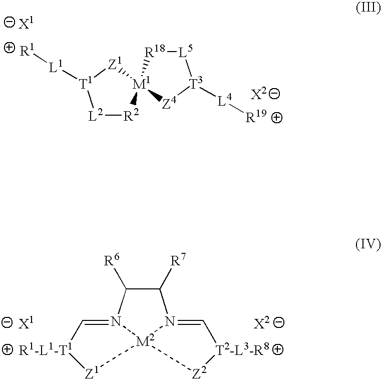
wherein
R¹ is a positively charged moiety which comprises a heteroatom and is chosen from a phosphonium derivative, a sulfonium derivative, an ammonium derivative and a positively charged heterocyclic ring, said phosphonium derivative, sulfonium derivative, ammonium derivative and said positively charged heterocyclic ring are unsubstituted or substituted with at least one substituent chosen from a halogen atom, —NO₂, —CN —OH, —CF₃ —COR^{S}, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl;
R² is CH═N—OH, PHR⁵, —C(═O)R³, —OH, —SH, —OR⁵, —SR⁵, —NH₂, —NHR⁴, —N(R⁴)₂, P(R⁵)₂, or —O(C═O)R³;
R³ is H, a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl;
R⁴ is a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for an amine;
R⁵ is a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for a hydroxy group, a phosphine, or a thiol group;
R⁶ and R⁷ are the same or different and each represent a hydrogen atom, C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, or C₁-C₁₂ heteroaryl;
R⁸ is a positively charged moiety which comprises a heteroatom and is chosen from a phosphonium derivative, a sulfonium derivative, an ammonium derivative, and a positively charged heterocyclic ring, said phosphonium derivative, sulfonium derivative, ammonium derivative and said positively charged heterocyclic ring are unsubstituted or substituted with at least one substituent chosen from a halogen atom, —NO₂, —CN —OH, —CF₃ —COR³, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl;
R¹⁸ is CH═N—OH, PHR⁵, —C(═O)R³, —OH, —SH, —OR⁵, —SR⁵, —NH₂, —NHR⁴, —N(R⁴)₂, P(R⁵)₂, or —O(C═O)R³;
R¹⁹ is a positively charged moiety which comprises a heteroatom and is chosen from a phosphonium derivative, a sulfonium derivative, an ammonium derivative, and a positively charged heterocyclic ring, said phosphonium derivative, sulfonium derivative, ammonium derivative and said positively charged heterocyclic ring are unsubstituted or substituted with at least one substituent chosen from a halogen atom, —NO₂, —CN —OH, —CF₃ —COR³, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl;
L¹ is a linker or a chemical bond;
L² is a linker or a chemical bond;
L³ is a linker or a chemical bond;
L⁴ is a linker or a chemical bond;
L⁵ is a linker or a chemical bond;
X¹ is an anion chosen from F⁻, Cl⁻, Br⁻, I⁻, ClO₄⁻, PF₆⁻, N₃⁻, BF₄⁻, SbF₆⁻, BH₄⁻, R³OSO₃⁻, (FSO₂)₂N⁻, (CF₃SO₂)₂N⁻, (C₂F₅SO₂)₂N⁻, (CF₃SO₂)₃C⁻, CF₃SO₃⁻, CF₃COO⁻, AsF₆⁻, CH₃COO⁻, (CN)₂N⁻, and NO₃⁻;
X² is an anion chosen from F⁻, Cl⁻, Br⁻, I⁻, ClO₄⁻, PF₆⁻, N₃⁻, BF₄⁻, SbF₆⁻, BH₄⁻, R³OSO₃⁻, (FSO₂)₂N⁻, (CF₃SO₂)₂N⁻, (C₂F₅SO₂)₂N⁻, (CF₃SO₂)₃C⁻, CF₃SO₃⁻, CF₃COO⁻, AsF₆⁻, CH₃COO⁻, (CN)₂N⁻, and NO₃⁻;
Z¹ is —OH, —SH, —OR⁵, —SR⁵, —NH₂, —NHR⁴, —N(R⁴)₂, —PHR³, —P(R³)₂;
Z² is —OH, —SH, —OR⁵, —SR⁵, —NH₂, —NHR⁴, —N(R⁴)₂, —PHR³, or —P(R³)₂;
Z⁴ is —OH, —SH, —OR⁵, —SR⁵, —NH₂, —NHR⁴, —N(R⁴)₂, —PHR³, or —P(R³)₂;
T¹ is a C₆-C₁₂ aryl, partially unsaturated C₁-C₁₂ heterocyclyl or C₁-C₁₂ heteroaryl;
T² is a C₆-C₁₂ aryl, partially unsaturated C₁-C₁₂ heterocyclyl or C₁-C₁₂ heteroaryl;
T³ is a C₆-C₁₂ aryl, partially unsaturated C₁-C₁₂ heterocyclyl or C₁-C₁₂ heteroaryl;
M¹ is a metal; and
M² is a metal;
said C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, and C₁-C₁₂ heteroaryl being unsubstituted or substituted with at least one substituent chosen from a halogen atom, —NO₂, —CN —OH, —CF₃ —COR^{S}, —SH, —OMe, —SMe, —SPh, —COOH, —COOR⁴, —NH₂, —NHR⁴, —N(R⁴)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl and C₁-C₁₂ heterocyclyl.

19. The complex of claim 18, wherein R¹ is a nitrogen-containing positively charged heterocyclic ring.

20. The complex of claim 18, wherein R¹ is of formula: 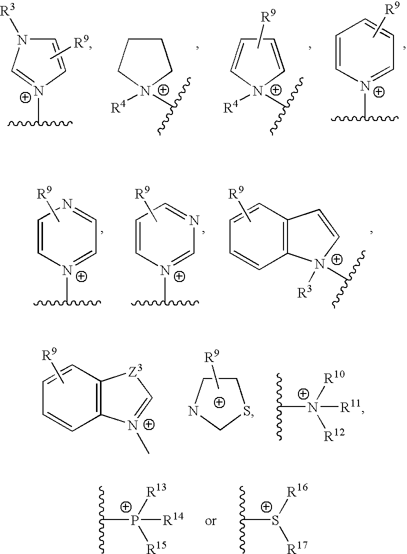
wherein
each formula is as presented above or substituted with at least one substituent as defined for R¹ in claim 18;
R⁹ represents a hydrogen atom, halogen atom, —NO₂, —CN —OH, —CF₃ —COR⁴, —SH, —OMe, —SMe, —SPh, —COOH, —COOR⁴, —NH₂, —NHR⁵, —N(R⁵)₂, C₂-C₂₀ alkenyl, C₁-C₂₀ alkoxy, C₁-C₂₀ alkyl, C₂-C₂₀ alkynyl, C₆-C₂₀ aralkyl, C₆-C₁₂ aryl, C₃-C₈ cycloalkyl, C₁-C₂₀ aminoalkyl, C₁-C₆ hydroxyalkyl, C₁-C₁₂ heteroaryl or C₁-C₁₂ heterocyclyl;
R¹⁰, R¹¹, and R¹² are same or different and each independently represent a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for an amine;
R¹³, R¹⁴, and R¹⁵ are same or different and each independently represent a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, or a suitable protecting group for a phosphorus atom;
R¹⁶ and R¹⁷ are same or different and each independently represent a C₁-C₂₀ alkyl, C₃-C₁₂ cycloalkyl, C₁-C₁₂ heterocyclyl, C₂-C₂₀ alkenyl, C₂-C₂₀ alkynyl, C₆-C₁₂ aryl, C₆-C₂₀ aralkyl, C₆-C₂₀ alkylaryl, C₁-C₁₂ heteroaryl, and a suitable protecting group for a sulphur atom;
Z³ is O or S; and
R³, R⁴, and R⁵ is as previously defined in claim 18.
